(19) United States
(12) Patent Application Publication
(10) Pub. No.: US 2021/0009914 A1
(43) Pub. Date: Jan. 14, 2021

(54) NOVEL APPLICATIONS OF PULSED ELECTRIC FIELD AND E-BEAM TECHNOLOGY

(71) Applicant: KEMIN INDUSTRIES, INC., DES MOINES, IA (US)

(72) Inventors: Liesbet Thijs, Kasterlee (BE); Raf Snoekx, Kalmthout (BE); Erwin Witters, Temse (BE); Alexandra Weallans, Marlborough (GB); Stefaan Van Dyck, Brasschaat (BE); Ingrid Somers, Kasterlee (BE)

(21) Appl. No.: 16/926,423

(22) Filed: Jul. 10, 2020

Related U.S. Application Data
(60) Provisional application No. 62/872,512, filed on Jul. 10, 2019.

(51) Int. Cl. C10L 5/44; A23L 5/30; C12N 1/06; C12N 13/00

(52) U.S. Cl. CPC C10L 5/44; C12N 13/00; C12N 1/066; A23L 5/30

(57) ABSTRACT

The invention describes antimicrobial treatment of animal feed, and other matrices with pulsed electric field (PEF) technology or e-beam technology combined with at least one antimicrobial and/or at least one surfactant. The use of this combined methodology approach results in a synergistic reduction of microbial load in the matrix of interest and shows bactericidal effects instead of bacteriostatic effects compared to the use of the technology alone. The addition of an antimicrobial agent in combination with the technology results in a long-lasting antimicrobial effect, preventing re-contamination, which cannot be achieved by using an energetic field alone. Furthermore, the invention describes treatment of the animal feed and other matrices with PEF or e-beam to increase nutrient digestibly of the matrix. Another aspect of the invention relates to providing a suitable alternative to heat treatment, or formaldehyde treatment, in order to decontaminate feed, human and pet food.

### CROSS-REFERENCE TO RELATED APPLICATIONS

This application claims the benefit of priority to U.S. Provisional Patent Application No. 62/872,512, filed Jul. 10, 2019, entitled “NOVEL APPLICATIONS OF PULSED ELECTRIC FIELD AND E-BEAM TECHNOLOGY,” the entire disclosure of which is incorporated herein in its entirety.

### BACKGROUND OF THE INVENTION

An energetic field (electric, magnetic) can be used to tackle microbiological contamination in many different markets. The use of this methodology can result in a significant reduction of microbial load in the matrix of interest, depending on process parameters, product parameters and microbial characteristics.

Pulsed electric field (PEF) technology is a non-thermal method of food preservation that uses short pulses of electricity and causes minimal detrimental effect on food quality attributes, in contrast to traditional thermal processing methods. Two key applications of PEF are cell disintegration for mass transfer enhancement and inactivation of microorganisms, with the matrix to be treated submerged in water. PEF technology involves the application of short voltage pulses (1-100 μs) at electric fields in the range of 0.1-80 kV/cm to liquid or semi-solid foods placed between two electrodes. More specifically, electric fields in the range of 0.1-1 kV/cm are used to induce stress in plant cells (reversible permeabilization); 0.5-3 kV/cm in the case of irreversible permeabilization of plant and animal tissue and a range of 15-40 kV/cm is used to result in irreversible permeabilization of microbial cells (Jäger, 2012) Gonzalez and Barret (2010) reported complete breakdown of microbial membranes at a field strength of 15 kV/cm. Yogesh et al., 2016 reported that the electric field intensity needs to above 10 kV/cm for irreversible electroporation to be occurred (Yogesh et al., 2016). However, higher field strengths between 10-80 kV/cm were reported by Bansal et al (2015) for pasteurization. (Bansal et al., 2015). Toepfl et al., 2014 reported that PEF generally employs high electric fields higher than 10 kV/cm for loss of vitality and microbial inactivation of e.g. Escherichia. coli (Toepfl et al., 2014). The mechanism by which microorganisms are inactivated by PEF is based on ion movement induced by the electric field resulting in an increase of the transmembrane potential, polarization of the membrane and finally resulting in pore formation in the membrane, which can be temporary or permanent depending on the intensity of the electric field (Toepfl et al., 2014). Generally speaking, the technology relates primarily to liquid or semi-solid products, but less to solid products.

In addition to the electric field strength of PEF technology, it must be noted that microbial inactivation also depends on characteristics of the matrix (e.g. water activity) as well as on microbial characteristics. Feed and its raw materials show very low water activity levels and moisture content (in general, feed has a water activity level of ˜0.6 and a moisture content of ˜12%). This low water activity level increases the resistance of microbial cells to PEF treatment, as well as to treatment with organic acids; due to reduction of the membrane permeability and fluidity. In a limited number of studies, the use of PEF for inactivation of microbial cells in matrices with low a_{w} and low moisture content has been described in dark rye flour (Keith et al., 1998b; (moisture content of 9.9-10.6%) and dried herbs (Keith et al., 1998a; moisture content of 5.2%). In these studies, only a very limited reduction in microbial load could be observed (i.e. 0.6-1 log reduction) although high field strengths were applied (i.e. 20-80 kV/cm). Also in beef burgers (Bolton et al. 2002; parameters not disclosed) and raw chicken meat (Clemente et al., 2020), no significant antimicrobial effect was observed. In this case, the authors suggested that the ineffectiveness of PEF on these matrices can be explained by the high protein content and lipid concentration as proteins can diminish the effect due to absorption of active radicals and ions resulting from the discharges.

In addition, PEF has been used in combination with other preservation methods such as the use of essential oils or organic acids to decontaminate meat (Bolton et al., 2002; Clemente et al., 2020) or bacteria in suspension (El Zakhem et al., 2010; Liu et al., 1997; Ait-Ouazzou et al., 2013). On meat, neither the combined use of essential oils nor the use of organic acids with PEF delivered a significant reduction in bacterial counts. In contrast, some synergistic effects could be observed, but only in liquid products and always at high field strengths (>12 kV/cm).

As indicated above, PEF is also being used in the industry as an extraction method for specific cell structures. Moreover, it has been reported that it can induce structural changes in waxy rice starch significantly affecting its digestibility (Zeng et al., 2016), although effects were mainly observed at a very high intensity of 50 kV/cm. Also in meat, higher protein digestibility values were reported but again only at high field strength of 10 kV/cm) (Bhat et al., 2018).

Electron beams are commonly used in industry for medical, environmental and material processing applications (Ozer, 2017). The costs of high intensity treatments make its implementation for feedstuff treatment non-economical. Different doses are required for various industrial processes covering a wide range (0.1 kGy to 1000 kGy). In terms of microbial growth control, dosages of less than 3 kGy are typically applied to control fungi, bacteria or parasites (Lung et al., 2015, Kashiwagi et al., 2012 and Cleland, 2009). The use of E-beam with antimicrobials such as sodium diacetate, potassium, lactate, and potassium benzoate has been reported to control growth of Listeria monocytogenes (gram positive bacteria) but only a bacteriostatic effect was reported and not a bactericidal effect (Sommers et al., 2003; Zhu et al., 2008).

The present inventors have determined that combining an energetic field (PEF or E-beam) with antimicrobial molecules and/or emulsifying agents/surfactants can increase the effectiveness of PEF/E-beam treatment while lowering the additional treatment costs due to the lower electric/magnetic fields used as a result of the use of antimicrobials and/or surfactants. Moreover, the use of an antimicrobial product (consisting of an antimicrobial and emulsifying agent/surfactant) has a long-lasting and synergistic antimicrobial effect, which cannot be achieved by use of PEF/E-beam alone. Furthermore, an improvement of nutrient digestibility of feed is demonstrated under the same test conditions as for microbial decontamination, resulting in desirable effects on both the feed safety and nutritional values of the feed and its raw materials and/or byproducts.

### SUMMARY OF THE INVENTION

The present invention relates to the use of pulsed field technology (PEF) or E-beam in combination with antimicrobials, and optionally surfactants, for lowering the concentration of bacteria and other microbes, and their metabolic products, in animal feed and other materials. The inventors have surprisingly determined that the unique combination of PEF or E-beam in conjunction with antimicrobials and/or surfactants synergistically reduce microbe concentration in the matrices to which it is directed. In certain embodiments, the PEF or E-beam can be used solely with an antimicrobial. In alternative embodiments, the PEF or E-beam can be used with an antimicrobial and optionally a surfactant. The created synergy reduces the voltage intensity needed to use the PEF, thereby reducing the overall cost of treatment as compared to use of PEF alone. Moreover, it was totally unexpected to observe significant effects on nutrient digestibility parameters by PEF used at low field strength.

### DETAILED DESCRIPTION OF THE INVENTION

The technological device generates an energy field to cause detrimental effects on microbiological cell components (this is true at high field strengths (i.e. 10-80 kV/cm). The use of antimicrobial in combination with PEF/E-beam shows synergism in decreasing the microbiological load in the feed matrix or in its raw materials. Furthermore, the optional inclusion of an emulsifying agent/surfactant provides stabilization of the antimicrobial agent at the cell membrane of the micro-organism of interest and results in a homogeneous spread of the antimicrobial on the matrix of interest. The stabilization at the cellular level could mean that the changes made to the microbial cell by the electric field are preserved for a longer time post the electric field exposure. This will potentiate the antimicrobial agent as they will penetrate longer and faster into the microbial cell and causing a profound antimicrobial effect. This effect also extends the amount of time that the microbial cell has an increased sensitivity to microorganisms. Moreover, in certain embodiments, the inclusion of an emulsifying agent/surfactant is key to maintain the antimicrobial effect in case of solid matrices.

The invention is primarily used to reduce microorganisms found in animal feed and/or animal feed raw materials and/or byproducts used in the feed industry, but may also be used to reduce microorganisms in food products and pet food (and/or food and pet food raw materials and/or byproducts used in these industries). The invention may be used to treat any type or source of contaminated feed/food/pet food.

As used herein, “pulsed electric fields” or “PEF” is defined as a non-thermal method of using short pulses of electricity for microbial inactivation while causing minimal detrimental effect to the attributes of the media to which it is applied. Electron beam technology is also appropriate for use in the invention, which is a process in which high-velocity electrons are concentrated into a narrow beam with a very high planar power density. The electric field may be applied in the form of exponentially decaying, square wave, bipolar, or oscillatory pulses and at ambient, sub-ambient, or slightly above-ambient temperature.

In one embodiment of the invention, the PEF technology is used with a treatment gap at least slightly larger than the maximum particle size(s) of the media to be treated. The voltage applied should range from about 0.1-80 kV/cm, with about 5 kV/cm being preferred.

In certain embodiments, the PEF technology is used in combination with one or more antimicrobials and one of more surfactants. The antimicrobial agent may be any known antimicrobial including but not limited to short chain fatty acids and their glycerides, medium chain fatty acids and their glycerides, long chain fatty acids and their glycerides, essential oils and other phenolic compounds, hydrosols and its components, formaldehyde, chelating agents, and antimicrobial peptides. The antimicrobial agent is preferably effective against food-borne microbes, such as Salmonella, Campylobacter, E. coli, Aspergillus spp, Listeria and their endotoxins. According to at least one embodiment, organic acids are most preferred for use as antimicrobial in the invention. According to certain embodiments, the antimicrobial includes a combination of one or more organic acids. In another embodiment, the antimicrobial agent is selected from the group consisting of formic acid, carboxylated compounds containing C1-C6, phenolic compounds and/or mixtures of the same. In one embodiment of the invention, the antimicrobial agent is used in an amount of 0.3% by weight of the feed but can range between 0.05-3.0% by weight of the feed.

In addition to or instead of the antimicrobial, one or more emulsifiers/surfactants may also be applied to the feed along with the PEF/electron beam technology. Suitable emulsifiers for this purpose include, but are not limited to, soya lecithin, glycerin monostearate, potassium stearate, calcium stearoyl lactylate (CSL), DATEM, glyceryl monostearate, mono propylene glycol, SPAN 80, SPAN 20, sodium stearoyl lactylate (SSL), Tween, sodium stearate, glycerol triacetate, sugar esters, non-dairy creamer, calcium stearate, polyglycerol polyricinoleate (PGPR), lecithin, mono and diglycerides, mono and diglyceride derivatives, polyglycerol esters (PGE), propylene glycol esters (PGMS), sucrose esters, sorbitan esters and polysorbates, polyethylene glycol (PEG) and its derivatives, polypropylene oxide-co-polyethylene oxide and copolymer type surfactants. According to at least one embodiment, one or more surfactants are selected from the group consisting of PEG and/or polypropylene oxide and poly(ethyleneoxide)-co-poly(propyleneoxide). According to at least one embodiment of the invention, the emulsifier is a glyceryl PEG ricinoleate. If included, the one or more emulsifier may optionally be used in an amount ranging from about 0.00001-3% by weight of the feed.

In a typical operational setting, the antimicrobial and surfactant can be applied before and/or after the PEF/electron beam technology. According to at least one embodiment, the antimicrobial and surfactant are applied before the PEF/electron beam which the researchers have observed as providing the greatest synergistic effect. In the case of feed production, the application site can be in the feed mill, including but is not limited to the mixer, as well as in the feeder of the mixer, the conditioner, the loading point of the raw materials or in case of a feed raw material producer, trader or feed processing plant, on site treatment can take place upon arrival or shipment of the raw material. The application may also be applied to the feed without a feed mill. The time duration between the application of the antimicrobials and technology can vary.

Target microorganisms in the matrix are gram negative bacteria (such as Salmonella, Campylobacter, E. coli), gram positive bacteria (such as B. cereus, Listeria), molds (such as Aspergillus spp.), yeasts and viruses. The metabolic products of the target microorganisms may also be damaged by synergistic use of antimicrobials and/or surfactants and the PEF/electron beam technology. The invention further provides the benefit of providing extended antimicrobial action and protects against reinfection for a significant length of time following treatment. Furthermore, an improvement of nutrient digestibility of feed could be observed under the same test conditions of PEF as for microbial decontamination.

At least one embodiment of the present invention relates to a method of treating animal feed and/or animal feed components and/or byproducts of the feed industry to achieve a reduction in microbial contamination comprising applying to the animal feed and/or animal feed components an energetic field, said energetic field being selected from the group consisting of one or more of pulsed electric fields (PEF) and E-beam and a composition comprising one or more antimicrobial agents. In certain embodiments, the energetic field is PEF applied at a voltage ranging from about 0.1-80 kV/cm. In alternative embodiments, the energetic field is E-beam applied at a voltage ranging from about 0.1-4 kGy.

According to at least one embodiment, the antimicrobial is an organic acid. In certain embodiments, the antimicrobial is an organic acid selected from the group consisting of formic acid, carboxylated compounds containing C1-C6, phenolic compounds and/or mixtures of the same.

According to at least one embodiment, the antimicrobial composition further comprises one or more surfactants. In certain embodiments, the one or more surfactants is combined with the antimicrobial agent prior to applying to the animal feed and/or animal feed components. In alternative embodiments, the one or more surfactant is added to the animal and/or animal feed components separately.

At least one embodiment of the present invention relates to reducing the contamination in animal feed and/or animal feed components. For instance, in certain embodiments, gram positive and/or gram negative bacteria, molds, yeast and/or viruses are targeted.

According to at least one embodiment of the present invention, the step of applying the energetic field to the animal feed and/or animal feed components occurs prior to applying the antimicrobial composition to the animal feed and/or animal feed components. In alternative embodiments, the step of applying the energetic field to the animal feed and/or animal feed components occurs after applying the antimicrobial composition to the animal feed and/or animal feed components.

According to at least one embodiment of the present invention, the time duration between the application of the antimicrobial(s) and/or surfactant(s) and the energetic field can vary with a minimum of about 0.1 s.

In at least one embodiment, the time duration of applying the energetic field can vary with a minimum of about 0.1 s.

According to at least one embodiment of the present invention, the method occurs in a feed mill, a feed raw material producer, a trader or feed processing plant as well as to a feed without a feed mill.

According to at least one embodiment of the present invention, the animal feed and/or animal feed components are protected against reinfection for an increased length of time following treatment. In certain embodiments, the potential of microorganisms to produce endotoxins is reduced. In certain embodiments, the surfactant prolongs sensitivity of microorganisms present in the animal feed and/or the animal feed components.

According to at least one embodiment of the present invention, the method of applying an electric field and a composition containing one or more antimicrobials, for instance an organic acid, results in a bactericidal effect instead of a bacteriostatic effect.

At least one embodiment of the present invention relates to a method for treating animal feed and/or animal feed components and/or byproducts of the feed industry to increase digestibility of the matrix applying to the animal feed and/or animal feed components and/or animal byproducts used in the animal feed industry: an energetic field, said energetic field being selected from the group consisting of one or more of pulsed electric fields (PEF) and E-beam, and optionally applying antimicrobials and/or surfactants. In certain embodiments, the energetic field, PEF, is applied at a voltage ranging from about 0.1-80 kV/cm. In certain embodiments, the energetic field, electron-beam is applied at a voltage ranging from about 0.1-4 kGy.

Another aspect of the present invention relates to a suitable alternative to heat treatments for decontaminating human or pet food and/or human or pet food contaminates. According to at least one embodiment of the present invention, human or pet food and/or human or pet food components and/or byproducts are treated to lower microbial contamination by applying to the food and/or food components an energetic field, said energetic field being selected from the group consisting of one or more of pulsed electric fields (PEF) and E-beam and applying one or more antimicrobials, and optionally one or more surfactant. The energetic field may be applied before or after application of the one or more antimicrobial.

According to at least one embodiment of the present invention, the human or pet food product includes but is not limited to be raw carcass of poultry and red meat, and raw seafood, and the further processed parts and mechanistically deboned materials from the above.

Another aspect of the present invention relates to methods for decontamination of meat slurry derived from poultry (chicken, turkey, duck, goose or mixture thereof), mechanically separated poultry (chicken, turkey, duck, goose or mixture thereof), poultry skin, liver, gizzard, hearts, viscera (chicken, turkey, duck, goose or mixture thereof), pork, beef, bison, deer, lamb, goat (skin, heart, liver, stomach, mechanically separated meat slurry or mixture thereof).

Additionally, wet pet food may derive benefit from the treatment prior, during or after retort. Semi-moist treats and any other pet food related foods or treats that contain sufficient moisture to conduct a current.

The following examples are offered to illustrate but not limit the invention. Thus, it is presented with the understanding that various formulation modifications as well as method of delivery modifications may be made and still are within the spirit of the invention.

### EXAMPLES

### Example 1

### Synergistic Effects of PEF Technology, Antimicrobials and Emulsifiers

### Materials and Methods

Feed materials. Pig feed, naturally contaminated with Enterobacteriaceae, was obtained from Feed Design Lab (the Netherlands). The naturally contaminated pig feed, not treated with an antimicrobial product, was diluted with mash broiler feed obtained from AVEVE until the required contamination level was obtained (Enterobacteriaceae contamination of 4 log cfu/g). After this dilution, the mash feed was mixed intensively to ensure a homogeneous contamination.

PEF treatments. PEF experiments were carried out using the PEFPilot™ System (220V, 50 Hz) at ELEA GmbH (Quakenbrück, Germany) at room temperature. The feed was stored at room temperature (20-25° C.) before treatment. An amount of 50 percent additional water (tap-water at room temperature), was added to the feed to prevent the formation of air bubbles, which increase the chance for a dielectric breakdown and arching. Before PEF treatment, the treatment chamber was filled with the feed, the feed was leveled and pressed using a plastic stick. The treatment chamber had a capacity of 250 g. Exponential decay pulses with width of 40 microseconds and frequency of 2 Hz were applied. An electric field strength of 5 kV/cm and specific energies of 12 (60 pulses), 120 (600 pulses) and 168 kJ/kg (840 pulses) were applied. The treatment chamber was rinsed after each treatment and pre-cooled in ice-water before new feed samples were added. Each PEF treatment consisted of at least two replicates. The temperature of the feed was measured just after the PEF treatment.

Application of antimicrobial products. The products were sprayed on a thin layer of the feed (portions of 300 g) by use of a nebulizer. The product Sal CURB® Ba Liquid (lot number: 1911110883) was dosed at 6 kg/T. Formic acid 85% (RM00672, lot 20000200403), the main component of Sal CURB Ba Liquid (i.e. 50% formic acid) was dosed at 3 kg/T and 6 kg/T. Sal CURB Ba Liquid also contains a mixture of different surfactants.

Test setups. The effect of the application of the antimicrobials alone on the naturally contaminated feed was tested in a separate experiment. In a first series of PEF experiments (first trial), the antimicrobial products were sprayed on the feed just after applying the PEF treatments, while for the second series of experiments (second trial) the antimicrobial products were applied on the feed before the PEF treatments.

Determination of total Enterobacteriacea level. The total Enterobacteriaceae level (TEC) of the feed samples not treated and treated with the antimicrobial products and/or PEF was determined at 24 h after PEF/product treatments. Three suspensions of each feed sample were prepared by mixing 10 g of the sample with 90 g of saline and by homogenizing the samples for 60 seconds in a Stomacher. RAPID' Enterobacteriaceae plates (Bio-Rad, 3564004) were inoculated using a spiral plate counter (Eddy Jet, IUL Instruments). The plates were incubated overnight at 37° C. for 24 h. After incubation, the colonies were counted manually. To obtain accurate results, the minimum number of colonies counted per plate was specified as 10 (a lower number gives less statistically significant results). Values below this limit, at the lowest sample dilution (100 cfu/g), were reported to a value equal to half of this limit (50 cfu/g). The colony-forming units were log-transformed prior to the statistical analyses. The microbiological data were analyzed using the Statgraphics Centurion XVI software (Statpoint Technologies, Inc., Virginia, USA). All data were subjected to one-way ANOVA and differences were separated using the least significant differences procedure. All statements of significance were based on a P-value less than 0.05, unless otherwise specified.

### Results

Effect of PEF technology alone. In two independent trials, no significant antimicrobial effect of PEF technology was observed on feed (process parameters: exponential decay pulses at 5 kV/cm with width of 40 microseconds and frequency of 2 Hz; specific intensities of 12 kJ/kg, 120 kJ/kg or 168 kJ/kg; added water level of 50%, room temperature under atmospheric conditions). In trial 1, even at the highest intensity, no significant reduction could be observed. On the contrary, a slight but significant increase in the level of Enterobacteriaceae was observed after the different PEF treatments (at low, medium and high energetic level) compared to the untreated control. (Table 1). In trial 2, no significant disinfection effect of PEF technology could be observed i.e. ˜0.3 log reduction (process parameters: 5 kV/cm; specific intensities of 120 kJ/kg; added water level of 50%, room temperature under atmospheric conditions) (Table 2)

TABLE 1
Effect of PEF treatment without any antimicrobial
product against Total Enterobacteriaceae count in
naturally contaminated broiler feed. (Trial 1).

| PEF | | Log | | Log |
|---|---|---|---|---|
| treatment | Treatment 1 | (cfu/g)¹ | Stdev | reduction |
| No | Control | 4.26 a | 0.08 | — |
| Low intensity level | Control | 4.65 b | 0.11 | −0.39 |
| Medium intensity level | Control | 5.09 c | 0.17 | −0.83 |
| High intensity level | Control | 4.63 b | 0.24 | −0.37 |

¹Reported values are means of six replicates. a-c Values within columns with different superscripts are significantly different (P < 0.05) according to one-way ANOVA followed by LSD.

TABLE 2
Effect of PEF treatment without any antimicrobial
product against Total Enterobacteriaceae count in
naturally contaminated broiler feed (Trial 2).

| | PEF | Log | | Log |
|---|---|---|---|---|
| Treatment | treatment | (cfu/g) | Stdev | reduction |
| Control | No | 5.09 a | 0.13 | — |
| Control | Medium intensity level | 4.83 a | 0.11 | 0.26 |
| Control | High intensity level | 4.76 a | 0.39 | 0.33 |

¹Reported values are means of six replicates. Values within columns with different superscripts are significantly different (P < 0.05) according to one-way ANOVA followed by LSD.

These results suggest that PEF, applied as a sole treatment, with these process parameters, is not lethal to the microbial cell. The researchers also observed that an increase in energy input from 12 kJ/kg to 168 kJ/kg did not improve the PEF performance.

Effect of product treatments alone. Similarly, in the first study, it was also demonstrated that the application of organic acids alone resulted in a limited reduction of Enterobacteriaceae (˜0.2-0.8 log reduction) in this test setup. Only for a higher dosage of 6 kg/T of feed, a statistically significant effect could be observed (˜0.8 log reduction), as can be expected in a dose response curve (Table 3).

TABLE 3
Effect of different antimicrobial product treatments against
Total Enterobacteriaceae (TEC: Total Enterobacteriaceae count,
cfu: colony forming units) in naturally contaminated broiler
feed. Untreated contaminated broiler feed served as control.

| | Log | | Log |
|---|---|---|---|
| Treatment | (cfu/g)¹ | Stdev | reduction |
| Control | 4.17 a | 0.41 | — |
| Formic acid @ 3 kg/T | 3.80 ab | 0.23 | 0.37 |
| Formic acid @ 6 kg/T | 3.33 b | 0.73 | 0.84 |
| Sal CURB Ba @ 6 kg/T | 3.94 a | 0.32 | 0.23 |

¹Reported values are means of six replicates. a-b Values within columns with different superscripts are significantly different (P < 0.05) according to one-way ANOVA followed by LSD.

Effect of combination of PEF technology and product treatment. The order of application (i.e. chemical treatment followed by PEF treatment, or vice versa) has a major impact on the effectiveness level. Application of PEF treatment followed by chemical treatment resulted in ˜0.2-0.3 log reduction in Enterobacteriaceae at low intensity level; 0.2-0.6 log reduction at medium intensity level and 0.6-1.2 log reduction at high intensity level, compared to the no-treatment control (Table 4).

TABLE 4
Effect of PEF treatments at 5 kV/cm and 12 (low), 120 (medium) and
168 (high intensity level) kJ/kg combined with and without different
antimicrobial product treatments against Total Enterobacteriaceae
(TEC: Total Enterobacteriaceae count, cfu: colony forming units)
in naturally contaminated broiler feed. Product treatments were applied
after PEF treatments. The untreated contaminated broiler feed mixture,
containing 50% additional water, served as control.

| | | | | Log |
|---|---|---|---|---|
| PEF | | Log | | reduc- |
| treatment | Treatment | (cfu/g)¹ | Stdev | tion |
| No | Control | 4.26 a | 0.08 | — |
| Low intensity | Control | 4.65 b | 0.11 | −0.39 |
| level | Formic acid @ 3 kg/T | 3.94 de | 0.09 | 0.32 |
| | Formic acid @ 6 kg/T | 4.03 ade | 0.34 | 0.23 |
| | Sal CURB Ba @ 6 kg/T | 3.97 de | 0.11 | 0.29 |
| Medium intensity | Control | 5.09 c | 0.17 | −0.83 |
| level | Formic acid @ 3 kg/T | 4.10 ad | 0.23 | 0.16 |
| | Formic acid @ 6 kg/T | 3.71 fg | 0.08 | 0.55 |
| | Sal CURB Ba @ 6 kg/T | 3.86 ef | 0.17 | 0.40 |
| High intensity | Control | 4.63 b | 0.24 | −0.37 |
| level | Formic acid @ 3 kg/T | 3.34 h | 0.14 | 0.92 |
| | Formic acid @ 6 kg/T | 3.06 i | 0.10 | 1.20 |
| | Sal CURB Ba @ 6 kg/T | 3.61 g | 0.36 | 0.65 |

¹Reported values are means of six replicates. a-i Values within columns with different superscripts are significantly different (P < 0.05) according to one-way ANOVA followed by LSD.

Application of chemical treatment followed by PEF treatment resulted in 1.2-2.8 log reduction at medium intensity level and 2.1-3.4 log reduction at high intensity level, compared to the no-treatment control. A ˜3.4-log reduction could be observed when the antimicrobial was added at a dosage of 6 kg/tonne, followed by PEF treatment at the highest intensity (Table 5). As such, the researchers observed a clear synergistic effect when the chemical treatment is followed by PEF treatment (as the antibacterial effect is far much higher than a ˜1.2 log reduction in case of an added effect).

TABLE 5
Effect of different antimicrobial product treatments combined with
and without PEF treatments at 5 kV/cm and 120 (medium) and 168 (high
intensity level) kJ/kg against Total Enterobacteriaceae (TEC: Total
Enterobacteriaceae count, cfu: colony forming units) in naturally
contaminated broiler feed. Product treatments were applied before
PEF treatments. The untreated contaminated broiler feed mixture,
containing 50% additional water, served as control.

| | PEF | Log | | Log |
|---|---|---|---|---|
| Treatment | treatment | (cfu/g)¹ | Stdev | reduction |
| Control | No | 5.09 a | 0.13 | — |
| Formic acid @ 3 kg/T | | 4.15 b | 0.19 | 0.94 |
| Formic acid @ 6 kg/T | | 3.55 c | 0.09 | 1.54 |
| Sal CURB Ba @ 6 kg/T | | 4.08 b | 0.06 | 1.01 |
| Control | Medium intensity | 4.83 a | 0.11 | 0.26 |
| Formic acid @ 3 kg/T | level | 3.58 c | 0.19 | 1.51 |
| Formic acid @ 6 kg/T | | 2.25 e | 0.66 | 2.84 |
| Sal CURB Ba @ 6 kg/T | | 3.48 c | 0.10 | 1.61 |
| Control | High intensity | 4.76 a | 0.39 | 0.33 |
| Formic acid @ 3 kg/T | level | 2.95 d | 0.73 | 2.14 |
| Formic acid @ 6 kg/T | | 1.70 f | 0.00 | 3.39 |
| Sal CURB Ba @ 6 kg/T | | 2.14 ef | 0.64 | 2.95 |

¹Reported values are means of six replicates. a-f Values within columns with different superscripts are significantly different (P < 0.05) according to one-way ANOVA followed by LSD.

### Example 2

### Synergistic Effects of PEF Technology, Antimicrobials and Emulsifiers; Improved Nutrient Digestibility

### Materials and Methods

Feed materials. Pig feed, naturally contaminated with Enterobacteriaceae, was obtained from Feed Design Lab (the Netherlands). The naturally contaminated pig feed, not treated with an antimicrobial product, was diluted with mash broiler feed obtained from AVEVE until the required contamination level was obtained (Enterobacteriaceae contamination of 4 log cfu/g). After this dilution, the mash feed was mixed intensively to ensure a homogeneous contamination.

Application of antimicrobial products. The products were sprayed on a thin layer of the feed (portions of 500 g) by use of a nebulizer. The product Sal CURB® Ba Liquid (lot number: 191111883) was dosed at 6 kg/T. Formic acid 85% (RM00672, lot 20000200403), the main component of Sal CURB Ba Liquid (i.e. 50% formic acid) was dosed at 3 kg/T and 6 kg/T. Sal CURB Ba Liquid also contains a mixture of different surfactants. After treatment, the samples were sent to ELEA GmbH (Quakenbrück, Germany) for PEF treatments.

PEF treatments. PEF experiments were carried out using the PEFPilot™ System (220V, 50 Hz) at ELEA GmbH (Quakenbrück, Germany) at room temperature. The PEF treatments were performed at 8 days after application of the antimicrobial products. The feed samples were stored at room temperature (20-25° C.) before PEF treatment. Each PEF treatment consisted of at least two replicates. Quantities of 50, 20 and 15 percent additional water (tap-water at room temperature), were added to the feed to prevent the formation of air bubbles, which increase the chance for a dielectric breakdown and arching. Before PEF treatment, the treatment chamber was filled with the feed, the feed was leveled and pressed using a plastic stick. The treatment chamber had a capacity of 250 g. Exponential decay pulses with width of 40 microseconds and frequency of 2 Hz were applied. An electric field strength of 5 kV/cm and a specific energetic level of 120 kJ/kg (600 pulses) was applied. The treatment chamber was rinsed after each treatment and pre-cooled in ice-water before new feed samples were added.

Test setups. The antimicrobial products were always applied on the feed before the PEF treatments. The different quantities of added tap-water were mixed homogeneously in the feed just before the PEF treatments. After the treatments, all feed samples were stored at room temperature.

Determination of total Enterobacteriacea level. The total Enterobacteriaceae level (TEC) of the feed samples not treated and treated with the antimicrobial products was determined at 10 days after product treatment. The TEC of the PEF treated samples was determined at 48 h after PEF treatment. Three suspensions of each feed sample were prepared by mixing 10 g of the sample with 90 g of saline and by homogenizing the samples for 60 seconds in a Stomacher. RAPID' Enterobacteriaceae plates (Bio-Rad, 3564004) were inoculated using a spiral plate counter (Eddy Jet, IUL Instruments). The plates were incubated overnight at 37° C. for 24 h. After incubation, the colonies were counted manually, as explained in the Internal Instruction. To obtain accurate results, the minimum number of colonies counted per plate was specified as 10 (a lower number gives less statistically significant results). Values below this limit, at the lowest sample dilution (100 cfu/g), were reported to a value equal to half of this limit (50 cfu/g). The colony-forming units were log-transformed prior to the statistical analyses. The microbiological data were analyzed using the Statgraphics Centurion XVI software (Statpoint Technologies, Inc., Virginia, USA). All data were subjected to one-way ANOVA and differences were separated using the least significant differences procedure. All statements of significance were based on a P-value less than 0.05, unless otherwise specified.

In vitro sugar release test. Control feed samples (without addition of antimicrobials) of the first experiments at ELEA GmbH were used for the in vitro sugar release test. In these experiments, an electric field strength of 5 kV/cm and specific energies of 12 (60 pulses) and 120 kJ/kg (600 pulses) were applied. After the PEF treatments, control feed samples were dried in an oven at 55° C. for 3 days and afterwards stored in the freezer (−20° C.) until analysis. After thawing, all feed samples were grinded through a 1 mm-sieve. A quantity of 0.5 g of the feed raw material was weight and transferred into a clean, plastic tube. An appropriate quantity of the enzyme solution (Xygest HT, lot 1907106001, xylanase activity of 4,000,000 U/g) was added to the test tubes. Sodium acetate buffer pH 5.0, 0.1 M was added to a total volume of 5 ml. The Xygest HT extract was added to the feed raw materials at a dosage of 20,000,000 U/kg. The tubes were incubated in a shaking incubator (IKA®KS4000 icontrol) at 40° C. for a period of 4 hours with continuous stirring (stirring rate=230 rpm). After incubation, the tubes were allowed to sit for a period of 5 minutes to allow settling of the solids. Aliquots of 4 ml of the liquid fractions of each tube were transferred to test tubes and placed in a boiling water bath for 10 minutes, then cooled and centrifuged (1650×g, 5 minutes). The supernatant was analysed for the concentration of reducing sugars according to the Somogyi-Nelson procedure, with glucose as standard. The amount of reducing sugars was expressed as μmol glucose equivalents per ml supernatant per minute. All analyses were performed in duplicate.

### Results

In this trial, it was demonstrated that addition of surfactants as well as using a single or a blend of organic acids is key when the additional water content is reduced (Table 6). By reducing the additional water content before the PEF treatments, the efficacy of formic acid at 3 kg/T was maintained (at a water addition of 15%) and even a higher antimicrobial effect could be observed at a water addition of 20%. The combined application of Sal CURB Ba Liquid at 6 kg/T and PEF, lowered the total Enterobacteriaceae level with 2.4 and 2.5 log in the contaminated feed mixtures, containing 20 and 15% additional water, respectively. When the feed was treated with 15% water before PEF treatment, a significant better effect was observed for Sal CURB Ba Liquid at 6 kg/T (log reduction of 2.5), compared to formic acid at 3 kg/T (log reduction of 1.4). As the concentration of formic acid in the blend is equal to the concentration of the single formic acid treatment in this experiment, it is clear that the addition of surfactants, and/or other organic acids, are key to maintain the antimicrobial effect.

TABLE 6
Effect of different antimicrobial product treatments combined
with PEF treatments at 5 kV/cm and 120 (medium intensity
level) kJ/kg against Total Enterobacteriaceae (TEC: Total
Enterobacteriaceae count, cfu: colony forming units, COS:
chitosan oligosaccharide) in naturally contaminated broiler
feed. Product treatments were applied before PEF treatments.
The untreated contaminated broiler feed mixtures, containing
50%, 20% and 15% additional water, served as controls.

| Additional | | | | | Log |
|---|---|---|---|---|---|
| water to | PEF | | Log | | reduc- |
| feed | treatment | Treatment | (cfu/g)¹ | Stdev | tion |
| 50% | No | Control | 4.42 a | 0.20 | — |
| | Medium | Control | 4.26 a | 0.38 | 0.16 |
| | intensity | Formic acid @ | 2.82 c | 0.11 | 1.60 |
| | level | 3 kg/T | | | |
| 20% | No | Control | 4.50 a | 0.13 | — |
| | Medium | Control | 3.69 b | 0.24 | 0.81 |
| | intensity | Formic acid @ | 2.30 d | 0.66 | 2.20 |
| | level | 3 kg/T | | | |
| | | Sal CURB Ba @ | 2.10 de | 0.44 | 2.40 |
| | | 6 kg/T | | | |
| 15% | No | Control | 4.27 a | 0.27 | — |
| | Medium | Formic acid @ | 2.83 c | 0.56 | 1.44 |
| | intensity | 3 kg/T | | | |
| | level | Sal CURB Ba @ | 1.80 e | 0.24 | 2.47 |
| | | 6 kg/T | | | |

¹Reported values are means of six replicates. a-e Values within columns with different superscripts are significantly different (P < 0.05) according to one-way ANOVA followed by LSD.

In Table 6, it is demonstrated that the use of surfactants, and/or a blend of different organic acids helps to maintain a successful PEF application compared to a single organic acid.

Besides microbial de-activation, PEF technology was also able to alter the fiber structure of the feed, under the lower studied intensities. Altering the fiber structure would allow enzymes like protease and/or amylase to significantly affect the digestibility of protein and/or starch, respectively. A higher nutrient digestibility rate results in a higher amount of energy available for the animal. The fiber structure alteration allowed by the PEF treatment permitted a higher nutrient release in common feed (raw materials). Such property of the PEF treatment will allow the use of (alternative) raw materials and/or byproducts, currently having a limited digestibility and nutritional impact in the animal. In a first test, a sugar release test was performed on feed, after PEF treatment.

As can be observed in Table 7, PEF treatment was able to release more sugars from the feed, compared to the untreated control. A positive response is of the PEF treatments at low and medium energetic level in terms of sugar release since 6% and 12% more sugars were found in the extract supernatants, respectively compared to the non-treated feed sample. The effect of the PEF treatment at low energetic level on the release of reducing sugars was of the same magnitude of the addition of 5 kg/T Xygest HT to the non-treated feed sample.

TABLE 7
Effect of PEF treatments at 5 kV/cm and 12 (low) and 120
kJ/kg (medium intensity level) without and with addition
of the xylanase Xygest HT on the release of reducing sugars
in the supernatants after digestion of the basal broiler
feed. The untreated broiler feed served as control.

| | | | | | Relative |
|---|---|---|---|---|---|
| | | | | | release of |
| | | | Release | | reducing |
| | | | of reducing | | sugars |
| PEF | | Dosage | sugars | | equiv- |
| Treatment | Treatment | (U/kg) | (μg/min ml) | Stdev | alents |
| No | Control | — | 3989 | 7 | 100 |
| | 5 kg/T | 20,000,000 | 4264 | 0 | 107 |
| | Xygest HT | | | | |
| Low | Control | — | 4219 | 15 | 106 |
| intensity | 5 kg/T | 20,000,000 | 4503 | 6 | 113 |
| level | Xygest HT | | | | |
| Medium | Control | — | 4459 | 19 | 112 |
| intensity | 5 kg/T | 20,000,000 | 4781 | 23 | 120 |
| level | Xygest HT | | | | |

### Example 3

### Bactericidal Effects of Electron-Beam Technology, Antimicrobials and Emulsifiers

### Materials and Methods

Feed materials. Basal broiler feed was obtained from Feed Design Lab (the Netherlands).

Artificially contamination of broiler feed with Salmonella typhimurium. The basal broiler feed was artificially inoculated with S. typhimurium at DIL (Deutsches Institut für Lebensmitteltechnik e.V.) in Quackenbrück (Germany). Salmonella typhimurium was selected as representative feed isolate. The Salmonella strain was grown, 24 hours at 37° C., on Tryptone Soya Agar (TSA) plates. The biomass on the plates was resuspended in saline. This was done by adding saline to the plates, homogenizing this mixture and collecting the mixture in a test tube. The saline containing the Salmonella was sprayed onto the feed. After homogenization, the contaminated mash feed was left for a week to stabilize.

Application of antimicrobial products. Sal CURB® Ba Liquid was sprayed on a thin layer of the feed (portions of 500 g) by use of a nebulizer. The product Sal CURB® Ba Liquid (lot number: 191111883) was dosed at 6 kg/T. The addition of Sal CURB Ba Liquid (at 6 kg/T) on the Salmonella contaminated feed was performed at DIL.

Electron Beam treatments. E-beam experiments were carried out at the facilities of DIL (Deutsches Institut für Lebensmitteltechnik e.V.) at Max Rubner Institute in Karlsruhe (Germany) using a linear electron accelerator (LINAC, type CIRCE III from Thomson-CSF/Linac Technologies S.A. (Orsay, France), 5-10 MeV acceleration energy, 10 kW beam power) and an electromechanical conveyor system. For E-beam treatment, five different intensities (2, 4, 6, 8 and 10 kGy) at 5 MeV were applied. While the samples were irradiated, the non-irradiated control samples were exposed to ambient temperature of the linear accelerator facility. Each E-beam treatment consisted of three replicates. After the treatment, the samples were shipped at ambient conditions to DIL (Quackenbrück, Germany) for microbiological analyses. The absorbed dose was measured using alanine dosimeter tablets and analyzed by an external company (A\tilde{e}rial, Illkirch-Graffenstaden, France).

Quantitative and Qualitative detection of Salmonella. The microbial analyses were performed at DIL (Quackenbrück, Germany). The levels of S. typhimurium in the feed samples were determined before and after the defined intensities of E-beam treatment and after 7 weeks of storage at room temperature. The detection limit of the assay was 10 cfu/g. Values below this limit were set for further calculation to 5 cfu/g. The data were subjected to one-way ANOVA and differences were separated using the least significant differences procedure. All statements of significance were based on a P-value less than 0.05, unless otherwise specified. The presence-absence analysis of Salmonella typhimurium was evaluated according to ISO 6579 standards. Each sample unit consisted of a 100 g from which an analytical unit weighing 25 g is sub-sampled for presence/absence testing.

### Results

Effect of product treatments followed by E-beam treatments on Salmonella. Table 8 shows the levels of Salmonella after the E-beam treatments of different intensities (0, 2, 4, 6, 8 and 10 kGy) in the artificially contaminated broiler feed treated with and without Sal CURB Ba Liquid. The untreated inoculated broiler feed had an average Salmonella contamination of 5.6 log. At E-beam treatment intensity of 4 KGy, reductions of a 3.3 log and 3.6 could be observed, without and with addition of Sal CURB Ba Liquid, respectively. Close to 5 log reduction of Salmonella typhimurium was achieved by E-beam intensity of above 6 kGy, where the counts were below the detection limit.

TABLE 8
Effect of E-beam treatments of different intensities (0,
2, 4, 6, 8 and 10 kGy) combined with and without Sal CURB Ba
Liquid against Salmonella (cfu: colony forming units)
in artificially contaminated broiler feed. Sal CURB Ba Liquid
was applied before electron beam treatments. The untreated
contaminated broiler feed served as control.

| | Irradiation | | | |
|---|---|---|---|---|
| | intensity | Log | | Log |
| Treatment | (kGy) | (cfu/g)¹ | Stdev | reduction |
| Control | 0 | 5.63 a | 0.04 | — |
| | 2 | 4.40 b | 0.22 | 1.23 |
| | 4 | 2.32 d | 0.15 | 3.31 |
| | 6 | 0.80 f | 0.17 | 4.83 |
| | 8 | 0.70 f | 0.00 | 4.93 |
| | 10 | 0.70 f | 0.00 | 4.93 |
| Sal CURB Ba @ 6 kg/T | 0 | 5.56 a | 0.11 | 0.07 |
| | 2 | 4.18 c | 0.03 | 1.45 |
| | 4 | 2.03 e | 0.23 | 3.60 |
| | 6 | 0.70 f | 0.00 | 4.93 |
| | 8 | 0.70 f | 0.00 | 4.93 |
| | 10 | 0.70 f | 0.00 | 4.93 |

¹Reported values are means of three replicates. a-f Values within columns with different superscripts are significantly different (P < 0.05) according to one-way ANOVA followed by LSD.

After 7 weeks of storage, Salmonella levels in the feed samples treated with E-beam (at an intensity of 0, 2 and 4 kGy) were counted again to investigate the effect of the treatments on the shelf life of the feed (Table 9) (to study either the bacteriostatic or bactericidal effect of E-beam in combination with antimicrobials and surfactants). After storage, an 0.7 log and 1.3 log reduction of Salmonella was observed for the untreated inoculated broiler feed and the feed treated with Sal CURB Ba Liquid, respectively. After an E-beam treatment intensity of 2 KGy, reductions of 1.2 log and 1.4 log were obtained, without and with addition of Sal CURB Ba Liquid, respectively. Higher reductions of Salmonella counts were shown after 48 days storage. The level of Salmonella in the feed samples treated with E-beam at 2 kGy decreased further with 1.7 log and 2.4 log, without and with addition of Sal CURB Ba Liquid, respectively and compared to the untreated broiler feed. Similar results were observed for the feed samples treated with E-beam at 4 kGy. At day 48, a reduction of almost 4.2 log in Salmonella count was observed for the E-beam treatment at 4 kGy combined with Sal CURB Ba Liquid. For this treatment, the Salmonella counts were below the detection limit after the storage period. As such, combinations of E-beam at low intensities and Sal CURB Ba Liquid were more effective in reducing and maintaining the Salmonella levels in feed during storage than either treatment alone.

TABLE 9
Effect of E-beam treatments of different intensities (0,
2 and 4 kGy) combined with and without Sal CURB Ba Liquid
against Salmonella (cfu: colony forming units) in artificially
contaminated broiler feed after 1 day and after 48 days
storage at room temperature. Sal CURB Ba Liquid was applied
before electron beam treatments. The untreated contaminated
broiler feed served as control.

| | Day 1 | | Day 48 | |
|---|---|---|---|---|
| Treatment | Log (cfu/g)¹ | Stdev | Log (cfu/g)¹ | Stdev |
| Control | 5.63 a | 0.04 | 4.89 a | 0.11 |
| 2 kGy | 4.40 b | 0.22 | 3.16 c | 0.22 |
| 4 kGy | 2.32 d | 0.15 | 1.00 e | 0.30 |
| Sal CURB Ba @ 6 kg/T | 5.56 a | 0.11 | 4.23 b | 0.22 |
| Sal CURB Ba @ 6 kg/T + | 4.18 c | 0.03 | 2.51 d | 0.30 |
| 2 kGy | | | | |
| Sal CURB Ba @ 6 kg/T + | 2.03 e | 0.23 | 0.70 e | 0.29 |
| 4 kGy | | | | |

¹Reported values are means of three replicates. a-e Values within columns with different superscripts are significantly different (P < 0.05) according to one-way ANOVA followed by LSD.

Application point in full scale liquid/emulsified feed/food processes: For low energy electric/electromagnetic fields the application point is chosen to achieve maximum accessibility to surface of the bulk of the feed/food product.

It should be appreciated that minor dosage and formulation modifications of the composition and the ranges expressed herein may be made and still come within the scope and spirit of the present invention.

Having described the invention with reference to particular compositions, theories of effectiveness, and the like, it will be apparent to those of skill in the art that it is not intended that the invention be limited by such illustrative embodiments or mechanisms, and that modifications can be made without departing from the scope or spirit of the invention, as defined by the appended claims. It is intended that all such obvious modifications and variations be included within the scope of the present invention as defined in the appended claims. The claims are meant to cover the claimed components and steps in any sequence which is effective to meet the objectives there intended, unless the context specifically indicates to the contrary.

The foregoing description has been presented for the purposes of illustration and description. It is not intended to be an exhaustive list or limit the invention to the precise forms disclosed. It is contemplated that other alternative processes and methods obvious to those skilled in the art are considered included in the invention. The description is merely examples of embodiments. It is understood that any other modifications, substitutions, and/or additions may be made, which are within the intended spirit and scope of the disclosure. From the foregoing, it can be seen that the exemplary aspects of the disclosure accomplish at least all of the intended objectives.

### REFERENCES

Ait-Ouazzou, A., Espina, L., Garcia-Gonzalo, D. and Pagán R. 2013. Synergistic combination of physical treatments and carvacrol for Escherichia coli O157:H7 inactivation in apple, mango, orange and tomato juices. Food control. 32, pp. 159-167
Bansal, V., Sharma, A., Ghanshyam, C., Singla, M. L., Kim, K. H., 2015. Influence of pulsed electric field and heat treatment on Emblica officinalis juice inoculated with Zygosaccharomyces bailii. Food & Bioprod. Process. 95, 146e154.
Bhat, Z. F., Morton, J. D., Mason, S. L., Bekhit, A.E.D.A. 2018. Pulsed electric field: role in protein digestion of beef Biceps Femoris. Innovative Food Science and emerging technologies. 50:132-138.
Bolton, D. J., Catarame, C., Byrne, C., Sheridan, J. J., McDowel, D. A. and Blair, I. S. 2002. Letters in applied microbiology, 34, pp. 139-143.
Cleland, M. R., Galloway, R. A. New York, IBA industrial Inc (2009).
Clemente, L., Condón-Abanto, S., Pedrós-Garrido, S., Whyte, P. and Lyne, J. G. 2020. Efficacy of pulsed electric fields and antimicrobial compounds for the inactivation of Campylobacter jejuni in liquids and raw chicken. Food Control 107: 10649.
El Zakhem, H., Lanoiselle, J. L., Lebovka, N., Nonus, M., Allah, H. and Vorobiev, E., 2010. HUM Engineering Journal, 9(1), pp. 1-8
Gonzalez, M. E., Barrett, D. M. (2010) Thermal, high pressure, and electric field processing effects on plant cell membrane integrity and relevance to fruit and vegetable quality. Journal of Food Science 75, 121-130.
Jäger H. 2012 Process performance analysis of pulsed electric field (PEF) food applications. Dissertation. Technischen Universität Berlin
Kashiwagi, M., Hoshi, Y. (2012). Electron beam processing system and its application. Japan, NHV Corporation, Vol. 75
Kim, B. H., Jang, A., Lee, S. O., Min., J. S. and Lee, M. 2004. Combined effect of electron-beam (beta) irradiation and organic acids on shelf life of pork loins during cold storage.
Keith, W. D. (1998a). Microbial reduction in flour and spice using pulsed electric fields. PhD Dissertation, University of Guelph.
Keith., W. D., Harris, L. J. and Griffiths M. W. 1998b. Reduction of bacterial levels in flour by pulsed electric fields. Journal of Food Process Engineering 21: 263-269.
Liu, X., Yousef, A. E., Chism, G. W. 1997. Inactivation of Escherichia coli O157:H7 by the combination of organic acids and pulsed electric field. Journal of food safety, 16, pp. 287-299.
Lung, H., Cheng, Y. C., Chang, Y-H, Huang, H-W, Yang, B. B., Wang, C-Y. (2015). Microbial decontamination of food by electron beam irradiation. Trends in Food Science and Technology. 44 (1): 66-78.
Ozer, Z. N. (2017). Electron beam irradiation processing for industrial and medical applications. EPJ Web of Conferences. 154:01019.
Siemer C., Heinz V. (2014) Chapter 8—Effect of high-intensity electric field pulses on solid foods. Emerging technologies for food processing (Second edition), p. 147-154.
Sommers, C., X. Fan, B. A. Niemira, and K. Sokorai. 2003. Radiation (gamma) resistance and postirradiation growth of Listeria monocytogenes suspended in beef bologna containing sodium diacetate and potassium lactate. J. Food Prot. 66:2051-2056.
Toepl S., Siemer C., Heinz V. (2014) Chapter 8—Effect of high-intensity electric field pulses on solid foods. Emerging technologies for food processing (Second edition), p. 147-154.
Yogesh K. pulsed electric field processing of egg products: a review. (2016) J Food Sci Technol. 53(4934-945.
Zhu, M. J., A. Mendonca, H. A. Ismail, and D. U. Ahn. 2008. Effects of irradiation on survival and growth of Listeria monocytogenes and natural microflora in vacuum-packaged turkey hams and breast rolls. Poult. Sci. 87:2140-2145.
Zeng, F., Gao, Q-Y., Han, Z., Zeng, X-a., Yu, S-j. 2016. Structural properties and digestibility of pulsed electric field treated waxy rice starch. Food chemistry. 194: 1313-1319.

1. A method of treating animal feed and/or animal feed components and/or byproducts of the feed industry to achieve a reduction in microbial contamination comprising applying to the animal feed and/or animal feed components:
an energetic field, said energetic field being selected from the group consisting of one or more of pulsed electric fields (PEF) and E-beam; and
a composition comprising at least one antimicrobial.

2. The method of claim 1 wherein the energetic field, said energetic field being PEF is applied at a voltage ranging from about 0.1-80 kV/cm.

3. The method of claim 1 wherein the energetic field, said energetic field being E-beam is applied at a voltage ranging from about 0.1-4 kGy.

4. The method of claim 1 wherein the antimicrobial is an organic acid.

5. The method of claim 5 wherein the antimicrobial is an organic acid selected from the group consisting of formic acid, carboxylated compounds containing C1-C6, phenolic compounds and/or mixtures of the same.

6. The method of claim 1 further comprising adding one or more surfactants to the animal feed and/or animal feed components.

7. The method of claim 1 wherein the antimicrobial composition further comprises one or more surfactants.

8. The method of claim 1 wherein gram positive and/or gram negative bacteria, molds, yeast and/or viruses are targeted.

9. The method of claim 1 wherein the step of applying the energetic field to the animal feed and/or animal feed components occurs prior to applying the antimicrobial to the animal feed and/or animal feed components.

10. The method of claim 1 wherein the step of applying the energetic field to the animal feed and/or animal feed components occurs after applying the antimicrobial composition to the animal feed and/or animal feed components.

11. The method of claim 1 wherein the time duration between the application of the antimicrobial(s) and/or surfactant(s) and the energetic field can vary with a minimum of about 0.1 s.

12. The method of claim 1 wherein the time duration of applying the energetic field can vary with a minimum of about 0.1 s.

13. The method of claim 1 wherein the method occurs in a feed mill, a feed raw material producer, a trader or feed processing plant as well as to a feed without a feed mill.

14. The method of claim 1 wherein the animal feed and/or animal feed components are protected against reinfection for an increased length of time following treatment.

15. The method of claim 1 wherein the potential of microorganisms to produce endotoxins is reduced.

16. The method of claim 1 wherein the surfactant prolongs sensitivity of microorganisms present in the animal feed and/or the animal feed components.

17. The method of claim 1 results in a bactericidal effect instead of a bacteriostatic effect.

18. A method of treating animal feed and/or animal feed components and/or byproducts of the feed industry to increase digestibility of the matrix comprising applying to the animal feed and/or animal feed components and/or animal byproducts used in the animal feed industry an energetic field, said energetic field being selected from the group consisting of one or more of pulsed electric fields (PEF) and electron-beam (E-beam).

19. The method of claim 18 wherein the energetic field, said energetic field being PEF is applied at a voltage ranging from about 0.1-80 kV/cm.

20. The method of claim 18 wherein the energetic field, said energetic field being E-beam is applied at a voltage ranging from about 0.1-4 kGy.

21. The method of claim 18 wherein the method occurs in a feed mill, a feed raw material producer, a trader or feed processing plant as well as to a feed without a feed mill.

22. A method of treating human or pet food and/or components and/or byproducts to reduce microbial contamination comprising applying to the human or pet food and/or components and/or byproducts:
an energetic field, said energetic field being selected from the group consisting of one or more of pulsed electric fields (PEF) and E-beam; and
a composition comprising at least one antimicrobial.

23. The method of claim 22, wherein the antimicrobial composition further comprises one or more surfactants.

24. The method of claim 22, wherein the human or pet food and/or components and/or byproducts include poultry and red meat, seafood and/or meat slurry.
